(19) United States
(12) Patent Application Publication
(10) Pub. No.: US 2005/0214760 A1
(43) Pub. Date: Sep. 29, 2005

(54) Biomarkers for detecting ovarian cancer

(75) Inventors: Daniel W. Chan, Clarksville, MD (US); Zhen Zhang, Dayton, MD (US); Alex J. Rai, Staten Island, NY (US)

Correspondence Address: EDWARDS & ANGELL, LLP, P.O. BOX 55874, BOSTON, MA, 02205, US

(73) Assignee: Johns Hopkins University, Baltimore, MD (US)

(21) Appl. No.: 10/500,838

(22) PCT Filed: Jan. 7, 2003
(86) PCT No.: PCT/US03/00531

§ 371 Date: Feb. 2, 2005

Related U.S. Application Data
(60) Provisional application No. 60/346,536, filed on Jan. 7, 2002.

(51) Int. Cl. C12Q 1/68; G01N 33/574

(52) U.S. Cl. 435/6; 435/7.23

(57) ABSTRACT

New biomarkers are provided that are useful for detecting cancer in a patient sample, particularly ovarian cancer. In a preferred aspect, methods for qualifying ovarian cancer status in a subject are provided that comprises measuring at least one of Markers I through VII in a sample from the subject, and correlating the measurement with ovarian cancer status.

The present application claims the benefit of U.S. provisional application No. 60/346,536, filed Jan. 7, 2002, which is incorporated by reference herein in its entirety.

### FIELD OF THE INVENTION

The invention provides inter alia for new biomarkers useful for measuring the ovarian cancer status of a subject.

### BACKGROUND OF THE INVENTION

The poor prognosis of ovarian cancer diagnosed at late stages, the cost and risk associated with confirmatory diagnostic procedures, and its relatively low prevalence in the general population together pose extremely stringent requirements on the sensitivity and specificity of a test for it to be used for screening for ovarian cancer in the general population. Despite more than a decade of effort in this direction, there is still not a cost effective screening test that satisfies these requirements. For example, the best characterized tumor marker, CA125, is negative in approximately 30-40% of stage I ovarian carcinomas and its levels are elevated in a variety of benign diseases. See T. Meyer et al., Br J Cancer (2000) 82(9):1535-8; P. Buamah, J. Surg Oncol (2000) 75(4):264-5; M K Tuxen, Cancer Treat Rev (1995) 21(3):215-45.

The identification of tumor markers suitable for the early detection and diagnosis of cancer holds great promise to improve the clinical outcome of patients. It is especially important for patients presenting with vague or no symptoms or with tumors that are relatively inaccessible to physical examination. Ovarian carcinoma represents one of such insidious and aggressive cancers. It is the most lethal gynecologic malignancy in women with 23,400 new cases and 13,900 deaths expected in 2001. E. Banks et al. Int. J Gynecol Center (1997) 7:425-38; D. M. Parkin et al., IARC Scientif (1992); R. T. Greenlee et al., CA Cancer J. Clin (2001) 51:15-37. Despite considerable effort directed at early detection, no cost effective screening tests have been developed and women generally present with disseminated disease at diagnosis. P. J. Paley, Curr Opin Oncol, (2001) 13(5); R. F. Ozols et al., Principles and Practice of Gyneologic Oncology, 3^{rd} ed. Philadelphia: Lippincott, Williams and Wilkins, 2000, pp.: 981-1057.

Currently, CA125 is the best characterized serological tumor marker for advanced epithelial ovarian cancers. However, its use as a population-based screening tool for early detection and diagnosis of ovarian cancer is hindered by its low sensitivity and specificity. N. D. MacDonald et al. Eur J. Obstet Gynecol Reprod Biol (1999) 82(2):155-7; I. Jacobs et al., Hum Reprod (1989) 4(1):1-12; I-M. Shih et al. Although pelvic and more recently vaginal sonography has been used to screen high-risk patients, neither technique has the sufficient sensitivity and specificity to be applied to the general population. N. D. MacDonald et al. Eur J. Obstet Gynecol Reprod Biol (1999) 82(2):155-7. Recent efforts in using CA125 in combination with additional tumor markers, in a longitudinal risk of cancer model, and in tandem with ultrasound as a second line test have shown promising results in improving overall test specificity, which is critical for a disease such as ovarian cancer that has a relatively low prevalence. R. P. Woolas et al., J Natl Cancer Inst (1993) 85(21):1748-51; R. P. Woolas et al., Gynecol Oncol (1995) 59(1):111-6; Z. Zhang et al., Gynecol Oncol (1999) 73(1):56-61; Z. Zhang et al; American Society of Clinical Oncology 2001; 2001 Annual Meeting (ASCO 2001) Abstract; S. J. Skates et al., Cancer (1995) 76(10 Suppl):2004-10; I. Jacobs et al., Br Med J (1993) 306(6884):1030-34; U. Menon et al., British Journal of Obstetrics and Gynecology (2000) 107(2):165-69; R. C. Bast et al. Ovarian Cancer: ISIS Medical Media Ltd., Oxford, UK (2001). However, it is still well recognized that there is a critical need for new serological tumor markers that individually or in combination with other markers or diagnostic modalities deliver the required sensitivity and specificity for early detection of ovarian cancer. R. C. Bast et al. Ovarian Cancer: ISIS Medical Media Ltd., Oxford, UK (2001).

### SUMMARY OF THE INVENTION

The present invention provides, for the first time, novel protein markers that are differentially present in the samples of human cancer patients and in the samples of control subjects. The present invention also provides sensitive and quick methods and kits that can be used as an aid for diagnosis of human cancer by detecting these novel markers. The measurement of these markers, alone or in combination, in patient samples provides information that a diagnostician can correlate with a probable diagnosis of human cancer or a negative diagnosis (e.g., normal or disease-free). All the markers are characterized by molecular weight. The markers can be resolved from other proteins in a sample by using a variety of fractionation techniques, e.g., chromatographic separation coupled with mass spectrometry, or by traditional immunoassays. In preferred embodiments, the method of resolution involves Surface-Enhanced Laser Desorption/Ionization (“SELDI”) mass spectrometry, in which the surface of the mass spectrometry probe comprises adsorbents that bind the markers.

In other preferred embodiments, comparative protein profiles are generated using the ProteinChip Biomarker System from patients diagnosed with ovarian serous carcinoma and from patients without known neoplastic diseases. A subset of biomarkers was selected based on collaborative results from supervised analytical methods. Preferred analytical methods include the Classification And Regression Tree (CART) (see, L. Breiman et al., Classification and /Regression Trees: Wadsworth & Brooks, Monterey, Calif. 1994), implemented in Biomarker Pattern Software V4.0 (BPS) (Ciphergen, Calif.), and the Unified Maximum Separability Analysis (UMSA) procedure(see Z. Zhang et al., Proc of Critical Assessment of Techniques for Microarray Data analysis, CAMDA 2000, Dec. 18-19, 2000, Durham, N.C.), implemented in ProPeak (3Z Informatics, SC).

In a preferred embodiment, the analytical methods are used individually and in cross-comparison to screen for peaks that are most contributory towards the discrimination between ovarian cancer patients and the non-cancer controls.

In another aspect, the biomarkers are purified (at least in part) and identified. The selected biomarkers, together with the tumor marker CA125, were evaluated individually and in combination through multivariate logistic regression.

In a preferred embodiment, identified biomarkers are used individually, in combinations thereof, and with or without C125. The identified biomarkers include, the proteins at peaks 9.2 kD, 54 kD and 79 kD. The 79 kD protein was found to correspond to transferrin, while the 9.2 kD protein was determined to be a fragment of the haptoglobin precursor protein. The third, 54 kD protein was identified as immunoglobulin heavy chain.

In other preferred embodiments, a plurality of the identified biomarkers are detected, preferably at least two of the biomarkers are detected, most preferably at least three of the biomarkers are detected. The most preferred markers are
the 79 kD (Marker VII ) protein corresponding to transferrin
the 54 kD (Marker V) protein corresponding to immunoglobulin heavy chain
the 9.2 kD (Marker II) protein corresponding to a fragment of the haptoglobin precursor protein, and;
correlating the detection of one or more protein biomarkers with a diagnosis of ovarian cancer, wherein the correlation takes into account the detection of one or more protein biomarkers in each diagnosis, as compared to normal subjects. Preferably, one or more protein biomarkers are used to diagnose ovarian cancer. See Example 1 which follows.

In a preferred embodiment, the identified biomarker is substantially homologous to the 79 kD (Marker VII ) protein corresponding to transferrin. Preferably the identified biomarker is about 80% homologous to transferrin, more preferably the identified biomarker is about 90% homologous to transferrin; most preferably the identified biomarker is about 95%, 97%, 98% and 99% homologous to transferrin.

In another preferred embodiment, the identified biomarker is substantially homologous to the 54 kD (Marker V) protein corresponding to immunoglobulin heavy chain. Preferably the identified biomarker is about 80% homologous to immunoglobulin heavy chain, more preferably the identified biomarker is about 90% homologous to immunoglobulin heavy chain; most preferably the identified biomarker is about 95%, 97%, 98% and 99% homologous to immunoglobulin heavy chain.

In a preferred embodiment, the identified biomarker is substantially homologous to the 9.2 kD (Marker II) protein corresponding to a fragment of the haptoglobin precursor protein. Preferably the identified biomarker is about 80% homologous to the haptoglobin precursor protein, more preferably the identified biomarker is about 90% homologous to the haptoglobin precursor protein; most preferably the identified biomarker is about 95%, 97%, 98% and 99% homologous to the haptoglobin precursor protein.

While the absolute identity of all of these markers is not yet known, such knowledge is not necessary to measure them in a patient sample, because they are sufficiently characterized by, e.g., mass and by affinity characteristics. It is noted that molecular weight and binding properties are characteristic properties of these markers and not limitations on means of detection or isolation. Furthermore, using the methods described herein or other methods known in the art, the absolute identity of the markers can be determined.

Preferred methods for detection and diagnosis of cancer comprise detecting at least one or more protein biomarkers in a subject sample, and; correlating the detection of one or more protein biomarkers with a diagnosis of cancer, wherein the correlation takes into account the detection of one or more biomarker in each diagnosis, as compared to normal subjects, wherein the one or more protein markers are selected from:

| | |
|---|---|
| Marker I: | having a molecular weight of about 8.6 kD |
| Marker II: | having a molecular weight of about 9.2 kD |
| Marker III: | having a molecular weight of about 19.8 kD |
| Marker IV: | having a molecular weight of about 39.8 kD |
| Marker V: | having a molecular weight of about 54 kD |
| Marker VI: | having a molecular weight of about 60 kD |
| Marker VII: | having a molecular weight of about 79 kD. |

wherein one or more protein biomarkers are used to diagnose cancer.

In a preferred method for detection and diagnosis of ovarian cancer, comprises detecting at least one or more protein biomarkers in a subject sample, wherein the protein markers are selected from:

| | |
|---|---|
| Marker I: | having a molecular weight of about 8.6 kD |
| Marker II: | having a molecular weight of about 9.2 kD |
| Marker III: | having a molecular weight of about 19.8 kD |
| Marker IV: | having a molecular weight of about 39.8 kD |
| Marker V: | having a molecular weight of about 54 kD |
| Marker VI: | having a molecular weight of about 60 kD |
| Marker VII: | having a molecular weight of about 79 kD |

and; correlating the detection of one or more protein biomarkers with a diagnosis of ovarian cancer, wherein the correlation takes into account the detection of one or more protein biomarkers in each diagnosis, as compared to normal subjects. Preferably, one or more protein biomarkers are used to diagnose ovarian cancer.

In other preferred embodiments, a plurality of the biomarkers are detected, preferably at least two of the biomarkers are detected, more preferably at least three of the biomarkers are detected, most preferably at least four of the biomarkers are detected. The most preferred markers are

| | |
|---|---|
| Marker II: | having a molecular weight of about 9.2 kD |
| Marker III: | having a molecular weight of about 19.8 kD |
| Marker VI: | having a molecular weight of 60 kD |
| Marker VII: | having a molecular weight of about 79 kD |

and; correlating the detection of one or more protein biomarkers with a diagnosis of ovarian cancer, wherein the correlation takes into account the detection of one or more protein biomarkers in each diagnosis, as compared to normal subjects. Preferably, one or more protein biomarkers are used to diagnose ovarian cancer.

In one aspect, the amount of each biomarker is measured in the subject sample and the ratio of the amounts between the markers is determined. Preferably, the amount of each biomarker in the subject sample and the ratio of the amounts between the biomarkers and known ovarian cancer markers is also determined to assess the stage of ovarian cancer. The most preferred markers are the 79 kD (Marker VII ) protein corresponding to transferrin; the 54 kD (Marker V) protein corresponding to immunoglobulin heavy chain; the 9.2 kD (Marker II) protein corresponding to a fragment of the haptoglobin precursor protein. Any one or combination of these markers can be used to differentiate between different stages of ovarian cancer. These markers can be used together with a known ovarian cancer biomarker such as C 125. See the examples which follow and Table 2.

In another aspect, preferably a single biomarker is used in combination with one or more known cancer biomarkers for diagnosing cancer, more preferably a plurality of the markers are used in combination with one or more known cancer markers for diagnosing cancer. Preferred known cancer markers are ovarian cancer markers for diagnosing ovarian cancer, such as CA 125. It is preferred that one or more protein biomarkers are used in comparing protein profiles from patients susceptible to, or suffering from cancer, such as ovarian cancer, with normal subjects.

Preferred detection methods include use of a biochip array. Biochip arrays useful in the invention include protein and nucleic acid arrays. One or more markers are immobilized on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

In another preferred method, data is generated on immobilized subject samples on a biochip array, by subjecting said biochip array to laser ionization and detecting intensity of signal for mass/charge ratio; and, transforming the data into computer readable form; and executing an algorithm that classifies the data according to user input parameters, for detecting signals that represent markers present in ovarian cancer patients and are lacking in non-cancer subject controls.

Preferably the biochip surfaces are, for example, ionic, anionic, comprised of immobilized nickel ions. comprised of a mixture of positive and negative ions, comprises one or more antibodies, single or double stranded nucleic acids, comprises proteins, peptides or fragments thereof, amino acid probes, comprises phage display libraries.

In other preferred methods one or more of the markers are detected using laser desorption/ionization mass spectrometry, comprising, providing a probe adapted for use with a mass spectrometer comprising an adsorbent attached thereto, and; contacting the subject sample with the adsorbent, and; desorbing and ionizing the marker or markers from the probe and detecting the deionized/ionized markers with the mass spectrometer.

Preferably, the laser desorption/ionization mass spectrometry comprises, providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

The adsorbent can for example be, hydrophobic, hydrophilic, ionic or metal chelate adsorbent, such as, nickel or an antibody, single- or double stranded oligonucleotide, amino acid, protein, peptide or fragments thereof.

In another embodiment, a process for purification of a biomarker, comprising fractioning a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers. Fractionation is monitored for purity on normal phase and immobilized nickel arrays. Generating data on immobilized marker fractions on an array, is accomplished by subjecting said array to laser ionization and detecting intensity of signal for mass/charge ratio; and, transforming the data into computer readable form; and executing an algorithm that classifies the data according to user input parameters, for detecting signals that represent markers present in cancer patients and are lacking in non-cancer subject controls. Preferably fractions are subjected to gel electrophoresis and correlated with data generated by mass spectrometry. In one aspect, gel bands representative of potential markers are excised and subjected to enzymatic treatment and are applied to biochip arrays for peptide mapping.

In another aspect one or more biomarkers are selected from: gel bands representing

| | |
|---|---|
| Marker I: | having a molecular weight of about 8.6 kD |
| Marker II: | having a molecular weight of about 9.2 kD |
| Marker III: | having a molecular weight of about 19.8 kD |
| Marker IV: | having a molecular weight of about 39.8 kD |
| Marker V: | having a molecular weight of about 54 kD |
| Marker VI: | having a molecular weight of about 60 kD |
| Marker VII: | having a molecular weight of about 79 kD |

Purified proteins for detection of ovarian cancer and/or generation of antibodies for further diagnostic assays are provided for. Purified proteins are selected from:

| | |
|---|---|
| Marker I: | having a molecular weight of about 8.6 kD; |
| Marker II: | having a molecular weight of about 9.2 kD; |
| Marker III: | having a molecular weight of about 19.8 kD; |
| Marker IV: | having a molecular weight of about 39.8 kD; |
| Marker V: | having a molecular weight of about 54 kD; |
| Marker VI: | having a molecular weight of about 60 kD; and |
| Marker VII: | having a molecular weight of about 79 kD. |

The invention further provides for kits for aiding the diagnosis of cancer, comprising: an adsorbent attached to a substrate, wherein the adsorbent retains one or more biomarker selected from:

| | |
|---|---|
| Marker I: | having a molecular weight of about 8.6 kD; |
| Marker II: | having a molecular weight of about 9.2 kD; |
| Marker III: | having a molecular weight of about 19.8 kD; |
| Marker IV: | having a molecular weight of about 39.8 kD; |
| Marker V: | having a molecular weight of about 54 kD; |
| Marker VI: | having a molecular weight of about 60 kD; and |
| Marker VII: | having a molecular weight of about 79 kD. |

Preferably, the kit comprises written instructions for use of the kit for detection of cancer and the instructions provide for contacting a test sample with the absorbent and detecting one or more biomarkers retained by the adsorbent.

The kit provides for a substrate which allows for adsorption of said adsorbent. Preferably, the substrate can be hydrophobic, hydrophilic, charged, polar, metal ions.

The kit also suitably provides for an adsorbent wherein the adsorbent is an antibody, single or double stranded oligonucleotide, amino acid, protein, peptide or fragments thereof.

Detection of one or more protein biomarkers using the kit suitably may be by mass spectrometry or immunoassays such as an ELISA.

In another embodiment, various compositions are provided to further aid in the diagnosis of ovarian cancer:
A composition comprising Marker I and one more biomarkers selected from Markers II, III, IV, V, VI, and VII.
A composition comprising Marker II and one more biomarkers selected from Markers I, III, IV, V, VI, and VII.
A composition comprising Marker III and at least one more biomarkers selected from Markers I, II, IV, V, VI, and VII.
A composition comprising Marker IV and at least one more biomarkers selected from Markers I, II, III, V, VI, and VII.
A composition comprising Marker V and at least one more biomarkers selected from Markers I, II, III, IV, VI, and VII.
A composition comprising Marker VI and one more biomarkers selected from Markers I, II, III, IV, V, and VII.
A composition comprising Marker VII and one more biomarkers selected from Markers I, II, III, IV, V, and VI.

Preferably each of the markers in the compositions is purified.

Other aspects of the invention are described infra.

### BRIEF DESCRIPTION OF THE FIGURES

FIG. 1: Representative spectrum obtained from SELDI analysis. Plasma sample was run on IMAC-Ni ProteinChip array. Upper panel shows a portion of the protein profile in spectrum view. Lower panel is same profile shown in pseudo-gel view.

FIGS. 2A -2B: ProPeak analysis of 67 samples. The UMSA component analysis module of ProPeak was used to project 67 samples on to a 3D space (non-cancer: green, cancer: red). (A) Projection using all peaks. (B) Projection using only seven selected peaks.

FIG. 3A-3C: Biomarker Patterns Software analysis of 67 samples. (A) Tree diagram shows that two peaks can be used to separate the patient data into non-cancer and cancer groups. Green squares indicate decision nodes, while terminal nodes are in shades of blue (non-cancer) and red (cancer), indicating classification into the two groups. (B) Sample composition of terminal nodes (blue: non-cancer, green: cancer), nodes are left to right, as numbered in the tree-diagram. (C) A graph depicting the cost value in relation to the number of terminal nodes.

FIG. 4: Pseudo-gel view of SELDI analysis of 67 plasma samples showing relative abundance of all markers in three panels: 6-10 kD, 15-45 kD, and 50-90 kD. Asterisks indicate markers of interest. Non-cancer samples (38) are shown above blue line, cancer samples (29) shown below.

FIG. 5: Schematic diagram of protein purification protocol.

FIG. 6: Protein Identification: Molecular weights of peptide fragments were measured by tandem mass spectrometry using Q-TOF. Data from the 9.2 kD candidate marker is shown above. Selected peaks were further analyzed by MS/MS fragmentation, as shown in the inset.

FIG. 7. ROC analysis based on all 80 patients to compare diagnostic performance of four biomarkers (9.2 kD, 54 kD, 60 kD, and 79 kD) individually and in combinations through logistic regression.

FIG. 8. Scatter plot showing that combination of biomarkers 60 kD and 79 kD complements CA125 in separating ovarian cancer from control patients. Dashed line indicates decision boundary of a possible linear classification function. Vertical line at CA125=35U/mL indicates recommended cutoff value for CA125.

FIG. 9. ROC analysis based on 68 patients with available CA125 values to compare diagnostic performance of a combination of biomarkers 60 kd) and 79 kD, CA125, and a diagnostic index combining the two biomarkers and CA125.

### DETAILED DESCRIPTION OF THE INVENTION

As discussed above, we now provide new biomarkers that can aid in the detection and assessment of cancer in a patient, particularly ovarian cancer.

The present invention is based in part upon, the discovery of protein markers that are differentially present in samples of human cancer patients and control subjects, and the application of this discovery in methods and kits for aiding a human cancer diagnosis. Some of these protein markers are found at an elevated level and/or more frequently in samples from human cancer patients compared to a control (e.g, women in whom human cancer is undetectable). Accordingly, the amount of one or more markers found in a test sample compared to a control, or the mere detection of one or more markers in the test sample provides useful information regarding probability of whether a subject being tested has human cancer or not.

The protein markers of the present invention have a number of other uses. For example, the markers can be used to screen for compounds that modulate the expression of the markers in vitro or in vivo, which compounds in turn may be useful in treating or preventing human cancer in patients. In another example, markers can be used to monitor responses to certain treatments of human cancer. In yet another example, the markers can be used in the heredity studies. For instance, certain markers may be genetically linked. This can be determined by, e.g., analyzing samples from a population of human cancer patients whose families have a history of human cancer. The results can then be compared with data obtained from, e.g., human cancer patients whose families do not have a history of human cancer. The markers that are genetically linked may be used as a tool to determine if a subject whose family has a history of human cancer is pre-disposed to having human cancer.

In another aspect, the invention provides methods for detecting markers which are differentially present in the samples of a human cancer patient and a control (e.g., women in whom human cancer is undetectable). The markers can be detected in a number of biological samples. The sample is preferably a biological fluid sample. Examples of a biological fluid sample useful in this invention include blood, blood serum, plasma, nipple aspirate, urine, tears, saliva, etc. Because all of the markers are found in blood serum, blood serum is a preferred sample source for embodiments of the invention.

In a preferred aspect, methods are provided for qualifying ovarian cancer status in a subject comprising:

measuring at least one biomarker in a sample from the subject, wherein the biomarker is selected from the group consisting of:

| | |
|---|---|
| Marker I: | having a molecular weight of about 8.6 kD |
| Marker II: | having a molecular weight of about 9.2 kD |
| Marker III: | having a molecular weight of about 19.8 kD |
| Marker IV: | having a molecular weight of about 39.8 kD |
| Marker V: | having a molecular weight of about 54 kD |
| Marker VI: | having a molecular weight of about 60 kD |
| Marker VII: | having a molecular weight of about 79 kD, and |

combinations of such Markers I through VII; and

correlating the measurement with ovarian cancer status.

Any suitable methods can be used to detect or measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy. Additionally, the terms “detect”, “detecting, “measure”, “measuring” include any of a wide range of analyses including quantifying, qualifying and the like.

As discussed in greater detail below, comparative protein profiles can be generated from patients diagnosed with ovarian serous carcinoma and from patients without known neoplastic diseases. A subset of biomarkers was selected based on collaborative results from two supervised analytical methods. The selected biomarkers, together with the tumor marker CA125, were evaluated individually and in combination through multivariate logistic regression. Specifically, we have shown that high-throughput protein profiling combined with effective use of bioinformatics tools offers a viable approach to screening for tumor markers. Briefly, a preferred system utilizes chromatographic arrays (e.g. ProteinChip Arrays) to assay the samples e.g. using SELDI (Surface Enhanced Laser Desorption/Ionization). Proteins bound to the arrays can be read e.g. in a ProteinChip Reader, a time-of-flight mass spectrometer. The new biomarkers as a panel have shown significant separating power between the control and the ovarian cancer patients in this study and are complementary to CA125.

Unless defined otherwise, all technical and scientific terms used herein have the meaning commonly understood by a person skilled in the art to which this invention belongs. The following references provide one of skill with a general definition of many of the terms used in this invention: Singleton et al, Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Marker” in the context of the present invention refers to a polypeptide (of a particular apparent molecular weight) which is differentially present in a sample taken from patients having human cancer as compared to a comparable sample taken from control subjects (e.g., a person with a negative diagnosis or undetectable cancer, normal or healthy subject).

As used herein, “substantially homologous” refers to a polypeptide with, at least about 70%, at least about 75%, at least about 80%, and at least about 85%, at least about 90%, or at least about 95% identity or greater to a known biomarker such as the 79 kD (Marker VII ) protein corresponding to transferrin; the 54 kD (Marker V) protein corresponding to immunoglobulin heavy chain; the 9.2 kD (Marker II) protein corresponding to a fragment of the haptoglobin precursor protein. Percent identity and similarity between two sequences can be determined using a mathematical algorithm (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

To determine the percent identity of two amino acid sequences, the sequences are aligned for optimal comparison purposes (e.g., gaps are introduced in one or both of a first and a second amino acid or nucleic acid sequence for optimal alignment and non-homologous sequences can be disregarded for comparison purposes). The percent identity between the two sequences is a function of the number of identical positions shared by the sequences, taking into account the number of gaps, and the length of each gap which need to be introduced for optimal alignment of the two sequences. The amino acid residues at corresponding amino acid positions are then compared. When a position in the first sequence is occupied by the same amino acid residue as the corresponding position in the second sequence, then the molecules are identical at that position (as used herein amino acid or “identity” is equivalent to amino acid or “homology”).

A “comparison window” refers to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

For example, the percent identity between two amino acid sequences can be determined using the Needleman and Wunsch algorithm (J. Mol. Biol. (48): 444-453, 1970) which is part of the GAP program in the GCG software package (available at http://www.gcp.com), by the local homology algorithm of Smith & Waterman (Adv. Appl. Math. 2: 482, 1981), by the search for similarity methods of Pearson & Lipman (Proc. Natl. Acad Sci. USA 85: 2444, 1988) and Altschul, et al. (Nucleic Acids Res. 27(17): 3389-3402, 1997), by computerized implementations of these algorithms (GAP, BESTFIT, FASTA, and BLAST in the Wisconsin Genetics Software Package (available from, Genetics Computer Group, 575 Science Dr., Madison, Wis.), or by manual alignment and visual inspection (see, e.g., Ausubel et al., supra). Gap parameters can be modified to suit a user's needs. For example, when employing the GCG software package, a NWSgapdna.CMP matrix and a gap weight of 40, 50, 60, 70, or 80 and a length weight of 1, 2, 3, 4, 5, or 6 can be used. Examplary gap weights using a Blossom 62 matrix or a PAM250 matrix, are 16, 14, 12, 10, 8, 6, or 4, while exemplary length weights are 1, 2, 3, 4, 5, or 6. The GCG software package can be used to determine percent identity between nucleic acid sequences. The percent identity between two amino acid or nucleotide sequences also can be determined using the algorithm of E. Myers and W. Miller (CABIOS 4:11-17, 1989) which has been incorporated into the ALIGN program (version 2.0), using a PAM120 weight residue table, a gap length penalty of 12 and a gap penalty of 4.

The phrase “differentially present” refers to differences in the quantity and/or the frequency of a marker present in a sample taken from patients having human cancer as compared to a control subject. For examples, a marker can be a polypeptide which is present at an elevated level or at a decreased level in samples of human cancer patients compared to samples of control subjects. Alternatively, a marker can be a polypeptide which is detected at a higher frequency or at a lower frequency in samples of human cancer patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in the human cancer patients' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of human cancer. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without human cancer. A control amount can be either in absolute amount (e.g., μg/ml) or a relative amount (e.g., relative intensity of signals).

“Probe” refers to a device that is removably insertable into a gas phase ion spectrometer and comprises a substrate having a surface for presenting a marker for detection. A probe can comprise a single substrate or a plurality of substrates. Terms such as ProteinChip®, ProteinChip® array, or chip are also used herein to refer to specific kinds of probes.

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Gas phase ion spectrometer” refers to an apparatus that measures a parameter which can be translated into mass-to-charge ratios of ions formed when a sample is volatilized and ionized. Generally ions of interest bear a single charge, and mass-to-charge ratios are often simply referred to as mass. Gas phase ion spectrometers include, for example, mass spectrometers, ion mobility spectrometers, and total ion current measuring devices.

“Mass spectrometer” refers to a gas phase ion spectrometer that includes an inlet system, an ionization source, an ion optic assembly, a mass analyzer, and a detector.

“Laser desorption mass spectrometer” refers to a mass spectrometer which uses laser as means to desorb, volatilize, and ionize an analyte.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker Br 1 from specific species such as rat, mouse, or human can be selected to obtain only those polyclonal antibodies that are specifically immunoreactive with marker Br 1 and not with other proteins, except for polymorphic variants and alleles of marker Br 1. This selection may be achieved by subtracting out antibodies that cross-react with marker Br 1 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Energy absorbing molecule” or “EAM” refers to a molecule that absorbs energy from an ionization source in a mass spectrometer thereby aiding desorption of analyte, such as a marker, from a probe surface. Depending on the size and nature of the analyte, the energy absorbing molecule can be optionally used. Energy absorbing molecules used in MALDI are frequently referred to as “matrix.” Cinnamic acid derivatives, sinapinic acid (“SPA”), cyano hydroxy cinnamic acid (“CHCA”) and dihydroxybenzoic acid are frequently used as energy absorbing molecules in laser desorption of bioorganic molecules.

Preferably, the sample is prepared prior to detection of biomarkers. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. For example, a K30 spin column (available from Princeton Separation, Ciphergen Biosystems, Inc., etc.) can be used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by gas phase ion spectrometry for the detection of markers.

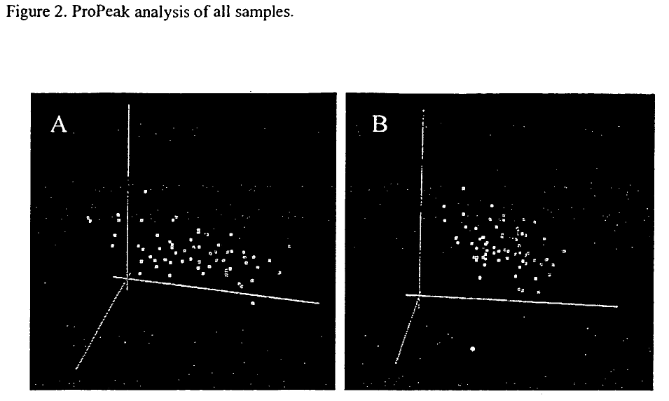

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's (see FIG. 2 and Example section below). Anion exchange chromatography allows separation of biomolecules in a sample that are more negatively charged from other types of biomolecules. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

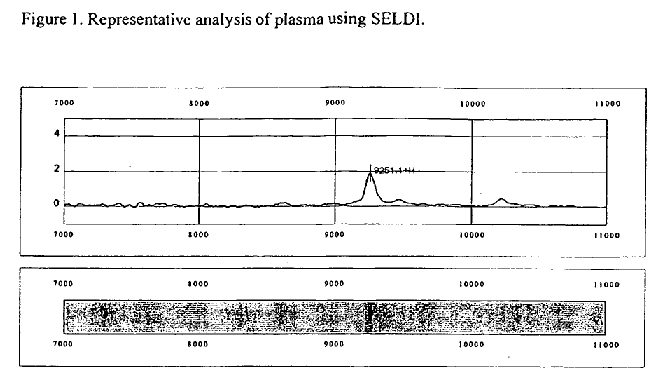

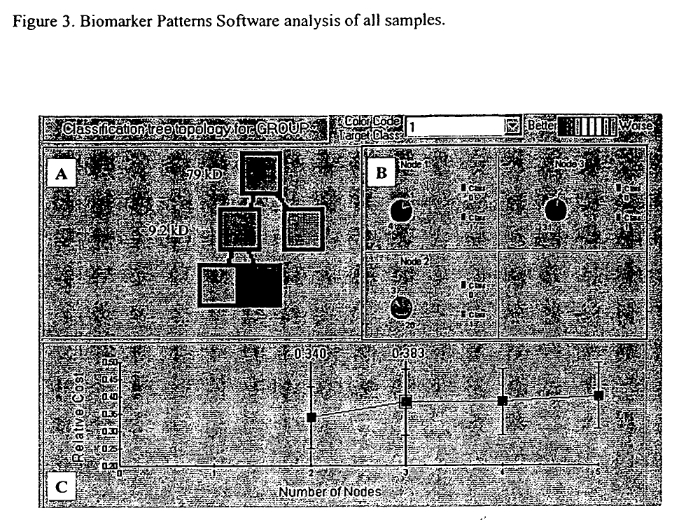

In yet another embodiment, a sample can be pre-fractionated by removing proteins that are present in a high quantity or that may interfere with the detection of markers in a sample. For example, in a blood serum sample, serum albumin is present in a high quantity and may obscure the analysis of markers. Thus, a blood serum sample can be pre-fractionated by removing serum albumin. Serum albumin can be removed using a substrate that comprises adsorbents that specifically bind serum albumin. For example, a column which comprises, e.g., Cibacron blue agarose (which has a high affinity for serum albumin) or anti-serum albumin antibodies can be used (see, e.g., FIGS. 1 and 3).

In yet another embodiment, a sample can be pre-fractionated by isolating proteins that have a specific characteristic, e.g. are glycosylated. For example, a blood serum sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Many types of affinity adsorbents exist which are suitable for pre-fractionating blood serum samples. An example of one other type of affinity chromatography available to pre-fractionate a sample is a single stranded DNA spin column. These columns bind proteins which are basic or positively charged. Bound proteins are then eluted from the column using eluants containing denaturants or high pH.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain proteins, and an eluant containing non-adsorbent proteins (i.e., proteins that did not bind to the first adsorbent) is collected. Then, the fraction is, exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Markers that do not bind to the first adsorbent is removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomolecules, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomolecules in a sample are separated by, e.g., isoelectric focusing, during which biomolecules in a sample are separated in a pH gradient until they reach a spot where their net charge is zero (i.e., isoelectric point). This first separation step results in one-dimensional array of biomolecules. The biomolecules in one dimensional array is further separated using a technique generally distinct from that used in the first separation step. For example, in the second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomolecules. Typically, two-dimensional gel electrophoresis can separate chemically different biomolecules in the molecular mass range from 1000-200,000 Da within complex mixtures.

Biomolecules in the two-dimensional array can be detected using any suitable methods known in the art. For example, biomolecules in a gel can be labeled or stained (e.g., Coomassie Blue or silver staining). If gel electrophoresis generates spots that correspond to the molecular weight of one or more markers of the invention, the spot can be further analyzed by gas phase ion spectrometry. For example, spots can be excised from the gel and analyzed by gas phase ion spectrometry. Alternatively, the gel containing biomolecules can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI (e.g., using ProteinChip® array) as described in detail below.

Prior to gas phase ion spectrometry analysis, it may be desirable to cleave biomolecules in the spot into smaller fragments using cleaving reagents, such as proteases (e.g., trypsin). The digestion of biomolecules into small fragments provides a mass fingerprint of the biomolecules in the spot, which can be used to determine the identity of markers if desired.

In yet another embodiment, high performance liquid chromatography (HPLC) can be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers. For example, the spots can be analyzed using either MALDI or SELDI (e.g., using ProteinChip® array) as described in detail below.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomolecules can be modified to improve detection resolution. For instance, neuraminidase can be used to remove terminal sialic acid residues from glycoproteins to improve binding to an anionic adsorbent (e.g., cationic exchange ProteinChip® arrays) and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of proteins. More recently, investigators are making use of protein biochips to capture and detect proteins. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomolecules in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, SAX-2, WCX-2, IMAC-3, LSAX-30, LWCX-30, IMAC-40, PS-10 and PS-20. Ciphergen's protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, SAX-2, WCX-2, IMAC-3, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The SAX-2 biochip has quarternary ammonium functionalities for anion exchange. The WCX-2 biochip has carboxylate functionalities for cation exchange. The IMAC-3 biochip has copper ions immobilized through nitrilotriacetic acid for coordinate covalent bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavadin and the like, to chip surfaces where they function to specifically capture analytes from a sample. The LSAX-30 (anion exchange), LWCX-30 (cation exchange) and IMAC-40 (metal chelate) biochips have functionalized latex beads on their surfaces. Such biochips are further described in: WO 00/66265 (Rich et al. (“Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000). U.S. patent application Ser. No. 09/908,518, filed Jul. 17, 2001 (“Latex Based Adsorbent Chip,” Pohl).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by appropriate means.

Analytes captured on the surface of a protein biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI technology is available from Ciphergen Biosystems, Inc., Fremont Calif. as part of the ProteinChip® System. ProteinChip® arrays are particularly adapted for use in SELDI. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

In another embodiment, an ion mobility spectrometer can be used to detect markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In yet another embodiment, a total ion current measuring device can be used to detect and characterize markers. This device can be used when the substrate has a only a single type of marker. When a single type of marker is on the substrate, the total current generated from the ionized marker reflects the quantity and other characteristics of the marker. The total ion current produced by the marker can then be compared to a control (e.g., a total ion current of a known compound). The quantity or other characteristics of the marker can then be determined.

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin; V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, antibodies that specifically bind to a marker can be prepared using any suitable methods known in the art. See, e.g, Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., women in whom human cancer is undetectable).

In another aspect, the invention provides methods for aiding a human cancer diagnosis using one or more markers, for example Markers I through VII. These markers can be used alone, in combination with other markers in any set, or with entirely different markers (e.g., CA 125 oncogene product) in aiding human cancer diagnosis. The markers are differentially present in samples of a human cancer patient, for example ovarian cancer patient, and a normal subject in whom human cancer is undetectable. For example, some of the markers are expressed at an elevated level and/or are present at a higher frequency in human cancer patients than in normal subjects. Therefore, detection of one or more of these markers in a person would provide useful information regarding the probability that the person may have human cancer.

Accordingly, embodiments of the invention include methods for aiding a human cancer diagnosis, wherein the method comprises: (a) detecting at least one marker in a sample, wherein the marker is selected from Marker I-VII; and (b) correlating the detection of the marker or markers with a probable diagnosis of human cancer. The correlation may take into account the amount of the marker or markers in the sample compared to a control amount of the marker or markers (up or down regulation of the marker or markers) (e.g., in normal subjects in whom human cancer is undetectable). The correlation may take into account the presence or absence of the markers in a test sample and the frequency of detection of the same markers in a control. The correlation may take into account both of such factors to facilitate determination of whether a subject has a human cancer or not.

Any suitable samples can be obtained from a subject to detect markers. Preferably, a sample is a blood serum sample from the subject. If desired, the sample can be prepared as described above to enhance detectability of the markers. For example, to increase the detectability of markers I, V, VII, a blood serum sample from the subject can be preferably fractionated by, e.g., Cibacron blue agarose chromatography and single stranded DNA affinity chromatography, anion exchange. chromatography and the like. Sample preparations, such as pre-fractionation protocols, is optional and may not be necessary to enhance detectability of markers depending on the methods of detection used. For example, sample preparation may be unnecessary if antibodies that specifically bind markers are used to detect the presence of markers in a sample.

Any suitable method can be used to detect a marker or markers in a sample. For example, gas phase ion spectrometry or an immunoassay can be used as described above. Using these methods, one or more markers can be detected. Preferably, a sample is tested for the presence of a plurality of markers. Detecting the presence of a plurality of markers, rather than a single marker alone, would provide more information for the diagnostician. Specifically, the detection of a plurality of markers in a sample would increase the percentage of true positive and true negative diagnoses and would decrease the percentage of false positive or false negative diagnoses.

The detection of the marker or markers is then correlated with a probable diagnosis of human cancer. In some embodiments, the detection of the mere presence or absence of a marker, without quantifying the amount of marker, is useful and can be correlated with a probable diagnosis of human cancer. For example, markers II, III, VI, can be more frequently detected in human ovarian cancer patients than in normal subjects. Thus, a mere detection of one or more of these markers in a subject being tested indicates that the subject has a higher probability of having a human cancer.

In other embodiments, the detection of markers can involve quantifying the markers to correlate the detection of markers with a probable diagnosis of human cancer. Thus, if the amount of the markers detected in a subject being tested is higher compared to a control amount, then the subject being tested has a higher probability of having a human cancer.

Similarly, in another embodiment, the detection of markers can further involve quantifying the markers to correlate the detection of markers with a probable diagnosis of human cancer wherein the markers are present in lower quantities in blood serum samples from human cancer patients than in blood serum samples of normal subjects. Thus, if the amount of the markers detected in a subject being tested is lower compared to a control amount, then the subject being tested has a higher probability of having a human cancer.

When the markers are quantified, it can be compared to a control. A control can be, e.g, the average or median amount of marker present in comparable samples of normal subjects in whom human cancer is undetectable. The control amount is measured under the same or substantially similar experimental conditions as in measuring the test amount. For example, if a test sample is obtained from a subject's blood serum sample and a marker is detected using a particular probe, then a control amount of the marker is preferably determined from a serum sample of a patient using the same probe. It is preferred that the control amount of marker is determined based upon a significant number of samples from normal subjects who do not have human cancer so that it reflects variations of the marker amounts in that population.

Data generated by mass spectrometry can then be analyzed by a computer software. The software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

In yet another aspect, the invention provides kits for aiding a diagnosis of human cancer, wherein the kits can be used to detect the markers of the present invention. For example, the kits can be used to detect any one or more of the markers described herein, which markers are differentially present in samples of a human cancer patient and normal subjects. The kits of the invention have many applications. For example, the kits can be used to differentiate if a subject has human ovarian cancer or has a negative diagnosis, thus aiding a human cancer diagnosis. In another example, the kits can be used to identify compounds that modulate expression of one or more of the markers in in vitro or in vivo animal models for human cancer.

In one embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample of blood serum is contacted on the probe. In another example, the kit may have instructions for pre-fractionating a sample to reduce complexity of proteins in the sample. In another example, the kit may have instructions for automating the fractionation or other processes.

In another embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of human ovarian cancer.

The following non-limiting examples are illustrative of the invention. All documents mentioned herein are fully incorporated herein by reference.

In the Example below, the following Materials and Methods were employed.

Samples.

A total of 80 specimens were used in this study. Blood samples were collected from 42 patients at the Johns Hopkins Hospital with sporadic ovarian serous neoplasms prior to tumor resection. These ovarian tumors included 11 FIGO-stage I,3 FIGO-stage II and 28 FIGO-stage III patients. The median age of these patients was 53 years (range: 36 to 84). Specimens from 38 women without known neoplastic diseases were used as controls. The median age of the controls was 57 years (range: 45 to 75). Specimens, collected in EDTA, Vacutainer tubes, were centrifuged at 2,000 rpm for 20 min and plasma samples were harvested to avoid leukocyte contamination. Specimens obtained prior to 2000 were analyzed for CA 125II using Centocor CA125II assays (Fujirebio Diagnostics, Malvern, Pa.). For the remaining specimens, CA125 levels were measured in either serum or EDTA plasma using the Tosoh AIA-PACK CA125 assay on the 600 II analyzer (Tosoh Medics, South San Francisco, Calif.). The Centocor CA125II assay is equivalent to the Tosoh CA125 assay (unpublished data). The Tosoh CA125 assay is approved for use in serum, however the assay was validated for plasma in house and results for serum and plasma were determined to be equivalent. Results were available in 68 out of the 80 total specimens. The median, mean and standard deviation of CA125 for the cancer group (n=32) were 58U/mL, 174.8U/mL, and 256.5U/mL, respectively, and for the control group (n=36), 7.6U/mL, 7.8U/mL, and 8.9U/mL, respectively. Among the total plasma samples (n=80), a group of 67 patients (29 ovarian cancer and 38 non-cancer cases) were initially analyzed for biomarker selection and identification. We then repeated the analysis on the entire collection of 80 specimens to include more early stage patients. Statistical analysis of biomarker performance was done based on the entire 80 patients.

ProteinChip® Analysis.

Fifteen microliters of each plasma sample was diluted into 25 ml 9 M urea, 2% CHAPS, and 50 mM Tris-HCl pH 9.0. Each sample was then diluted 1:40 in phosphate buffered: saline (PBS) pH 7.4, 50% acetonitrile (ACN) in dH2O, or 50 mM Na2HPO4 pH 6.0 for use with immobilized metal affinity capture type 3 (IMAC3), reverse phase (H4), or strong anion exchange type 2 (SAX2) 8-spot arrays respectively. IMAC3 ProteinChips were pretreated with nickel sulfate as per manufacturer's instructions. Using a bioprocessor, each array was then pre-washed in the appropriate wash buffer: PBS pH 7.4, 50% ACN in dH2O, and 100 mM Na2HPO4 pH 6.0 for IMAC, H4 and SAX2 respectively. Fifty μL of each sample was applied to each array type and incubated on a shaker for 40 minutes at room temperature. Samples were washed using 100 μl PBS pH 7.4, 100 μl 50% ACN in dH2O, and 100 μl 50 mM Na2HPO4 for IMAC, H4, and SAX2 respectively, repeated twice, followed by two quick rinses in dH2O. After air-drying, sinapinic acid (SPA), prepared as per manufacturer's instructions, was applied to each spot. The arrays were analyzed on a PBS-II mass reader (Ciphergen Biosystems, Fremont, Calif.) using SELDI 2.1b software (Ciphergen Biosystems, Fremont, Calif.). Data was collected by averaging 60 laser shots with an intensity of 240 and a detector sensitivity of 8.

Bioinformatics and Statistics.

The Ciphergen ProteinChip software system was used to identify qualified peaks from the raw spectrum data by applying a threshold to peak intensities that had been normalized against total ion current. Since more sophisticated procedures were used for the final peak selection, the initial threshold was set to capture the largest number of candidate peaks. Logarithmic transformation was applied to the data when needed to reduce peak intensity ranges. The final result is an m (peaks) by n (specimens) matrix, where an entry at row i column j presents the normalized relative abundance of proteins at mass weight corresponding to peak i in specimen j. Two supervised pattern classification methods, the Classification And Regression Tree (CART) (Breiman L, Friedman, J. H., Olshen R. A., and Stone, C. J. Classification and Regression Trees. Wadsworths & Brooks, Monterey, Calif.; 1984), implemented in Biomarker Pattern Software V4.0 (BPS) (Ciphergen, Calif.), and the Unified Maximum Separability Analysis (UMSA) procedure;(Zhang Z, Page, G., Zhang, H. Applying Classification Separability Analysis to Microarray Data, in Proc. of Critical Assessment of Techniques for Microarray Data Analysis. CAMDA '00, Dec. 18-19, 2000, Durham, N.C. 2000), implemented in ProPeak (3Z Informatics, SC), were used individually and in cross-comparison to screen for peaks that are most contributory towards the discrimination between ovarian cancer patients and the non-cancer controls. The UMSA algorithm as implemented in ProPeak is a linear classifier while the CART algorithm in BPS is a binary decision tree-type nonlinear classifier. In general the ranking and selection of peaks based on linear classification tend to be more robust, especially with the inherent variances and noise in the raw spectrum data. On the other hand, a non-linear classifier might give a better classification result even though extra caution needs to be exercised to avoid over-fitting data with superfluous biomarkers. The apparent consistency between results from these two approaches on our data provides additional confidence that the selected peaks reflect pathophysiological changes rather than artifactual differences.

The Classification and Regression Tree (CART) procedure constructs a binary decision tree that recursively partitions a given dataset into blocks of predicted positive and negative samples. The procedure minimizes a cost function that balances prediction errors and the total number of markers used. The relative importance of a peak is measured by the order in which it was selected in the decision tree and the number of correct predictions it is credited for.

Support vector machine (SVM) (Vapnik V N. Statistical Learning Theory. John Wiley & Sons, New York, 1998 ) has been applied to a number of biological expression data processing applications (Brown M, Grundy, W N, Lin, D, Cristianini, N, Sugnet, C W, Furey, T S, et al. Knowledge-based analysis of microarray gene expression data by using support vector machines. Proc Natl Acad Sci USA 2000;97:262-67). The UMSA procedure modifies the SVM learning algorithm to allow for the incorporation of data distribution information. For data sets with a small sample size relative to the number of variables, UMSA tends to be less sensitive than the typical SVM to possible labeling errors in data, such as those resulting from specimen contamination or misdiagnosed cases. Currently, ProPeak offers two analytical modules. The first is a UMSA component analysis module, which projects the original specimen as individual points into a three-dimensional component space. The components (axes) are linear combinations of the original spectrum peaks determined such that two pre-specified groups of data achieve maximum separability. The results can then be viewed in an interactive 3D display. The second module in ProPeak uses a backward stepwise process to compute a significance score to rank individual markers according to their collective contribution towards the separation of two groups of specimens under UMSA.

The peaks selected by BPS and UMSA analysis were evaluated individually, and in combinations of multiple peaks for their diagnostic performance using multivariate logistic regression. Diagnostic performance was assessed by estimating sensitivity and specificity, and using the area under the curve from receiver operating characteristic (ROC) curve analysis. For specimens with available CA125 values, results were compared to the diagnostic performance of CA125.

Biomarker Identification.

Based on the relative expression levels of the candidate biomarkers of interest within the plasma samples, a subset of samples were chosen to be used in protein purification. Plasma samples, 27 μL each, were first buffer-exchanged into 20 mM Tris-HCl, pH 9.0 buffer using K-30 size-selection spin columns (Ciphergen Biosystems, Fremont, Calif.) equilibrated with the same buffer. Proteins were then fractionated on anion-exchange spin columns based on their isoelectric point (pI). Each sample was applied to a spin microcolumn containing 100 μL of Q HyperD anion-exchanger resin (BioSepra), equilibrated in 20 mM Tris-HCl, pH 9.0 buffer. After binding, the flow through fraction was collected. Subsequent fractions were collected using 100 μL of pH 9.0 buffer and buffers at decreasing pH 8 (20 mM Tris-HCl), 20 mM phosphate/citrate combination buffers of pH 7.0, 6.0, 5.0, 4.0 and 3.0 buffers. Finally, columns were washed in an organic buffer containing 16.7% isopropanol, 33.3% acetonitrile, 0.1% trifluoroacetic acid, to remove the remaining proteins. Fractionation was monitored on both NP (normal phase) and IMAC-Ni (immobilized nickel array) arrays. An aliquot of 1 μL (of 120 μl total) of each fraction was applied to each spot on the NP (Normal Phase) array and 2 μl were used for each spot on the IMAC-Ni array. The ProteinChip reader (PBS II) was used to detect proteins in each spot of the array through automatic data acquisition mode at fixed laser intensity. The mass spectrometric profiles (intensity vs. M/z) of all plasma samples were compared to identify fractions containing the biomarkers of interest, as well as the purity of each biomarker. After identifying the fractions of interest, samples were separated by SDS-PAGE. A 16% acrylamide Tris-Glycine gel (Invitrogen/Novex) was used to isolate the 7 to 12 kD proteins, a 4-20% acrylamide Tris-Glycine gel was used for the 15 to 50 kD proteins and a 6% acrylamide Tris-Glycine gel was used for the 52 to 80 kD proteins. Gels were stained with Colloidal Blue (Invitrogen/Novex) and destained with deionized water. By correlating the mass spectra and Coomassie stained protein bands for high and low abundance proteins, we were able to identify the particular protein bands of interest. These were subsequently punched out using a disposable Pasteur pipette. The gel slices were destained and then the purified proteins in the gel slices were digested with 10 μL of 0.02 μg/μL modified trypsin in 25 mM ammonium bicarbonate, pH 8.0 buffer. Peptides generated by in-gel tryptic digestion were profiled using NP and H4 (hydrophobic) arrays. 1-2 μL of each digest was applied to each spot on the array, proteins were allowed to concentrate to dryness before 0.5 μL of 20% saturated cyano-4-hydroxycinnamic acid (CHCA) in 50%o acetonitrile, 0.5% TFA solution was applied to each spot. After the arrays were completely dry, the ProteinChip reader (PBS II) was used for peptide mapping. Peptide standards were used to internally calibrate the MS spectra for accurate peptide mass determination, and those obtained from control samples (trypsin incubated with blank gel plugs) were subtracted from the peptide maps. Subsequently, peptide masses were used for database searching and protein identification using ProFound (Rockefeller University) and MASCOT (MatrixScience). Protein identity was further confirmed by sequencing selected peptides from the tryptic digest using a ProteinChip interface PCI-1000 (Ciphergen, Fremont, Calif.) coupled to a Q-TOF II MS/MS (MicroMass, UK).

### EXAMPLE 1

Mass spectra of the initial group of 67 patients (cancer n=29, non-cancer n=38) were obtained from SELDI analysis using IMAC-Ni ProteinChips. FIG. 1 shows a representative view of the spectra showing proteins retained on the chip, in both spectrum and pseudo-gel view. Spectra of the 67 samples were analyzed using two bioinformatics software packages, Biomarker Pattern Software V4.0 (BPS) (Ciphergen, CALIF.), and ProPeak (3Z Informatics, SC).

Results were cross-compared in order to select a subset of peaks that possessed the maximum discriminatory power. Using the UMSA component analysis module in ProPeak, we were able to project the patient data onto a 3D space in which the cancer and non-cancer patients were best separated. (FIG. 2A). Subsequently, using the backward stepwise peak selection module, we selected seven peaks (8.6 kD, 9.2 kD, 19.8 kD, 39.8 kD, 54 kD, 60 kD, and 79 kD), for further analysis. Among them, peaks at 9.2 kD, 19.8 kD, and 60 kD showed higher expression levels on average among the specimens from the cancer patients compared to the controls while the remaining peaks demonstrated the inverse expression pattern. We then reapplied the UMSA component analysis using only these seven peaks to test whether they retained most of the discriminatory power of the original fill spectrum (FIG. 2B).

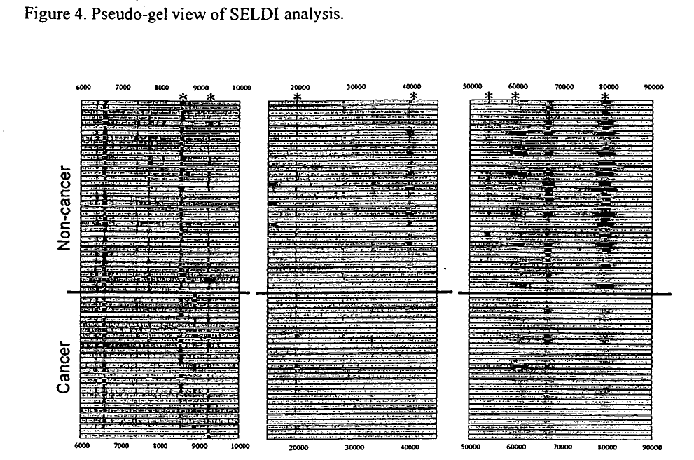

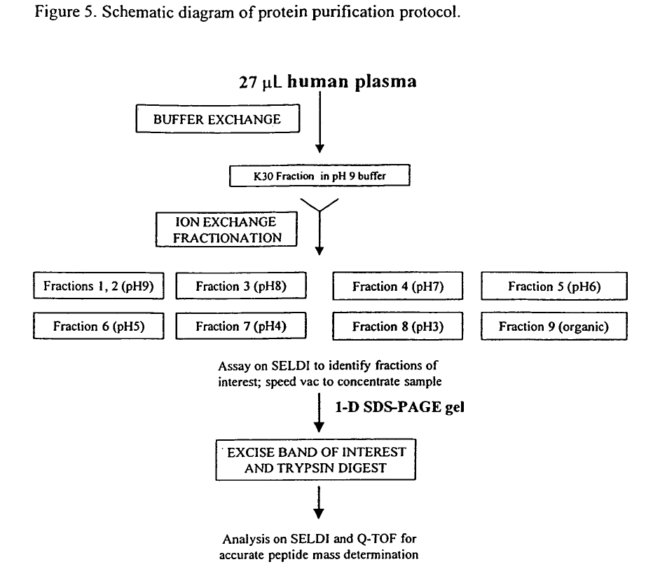

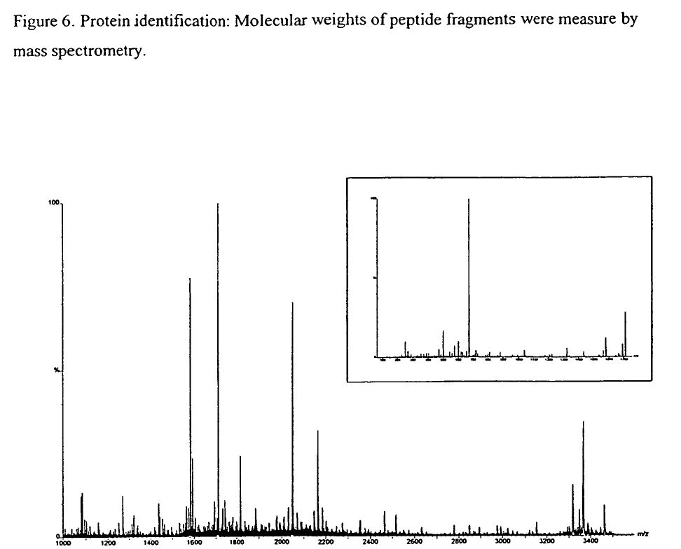

Using BPS, the peaks at 79 kD and 9.2 kD were identified as providing the optimal classification rate for the dataset (see FIG. 3). Compared to the results from ProPeak, these two peaks were ranked number 1 and 6, respectively. The pseudo-gel view of the seven selected protein peaks are given in FIG. 4. We were only able to purify and identify three proteins at peaks 9.2 kD, 54 kD and 79 kD. The flow diagrams describe the steps in protein purification (FIG. 5) and identification using tandem mass spectrometry (FIG. 6). The 79 kD protein was found to correspond to transferrin, while the 9.2 kD protein was determined to be a fragment of the haptoglobin precursor protein. The third, 54 kD protein was identified as immunoglobulin heavy chain.

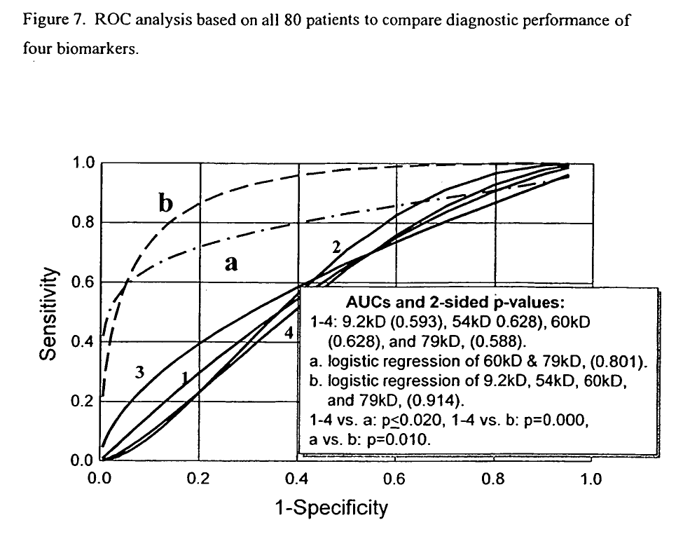

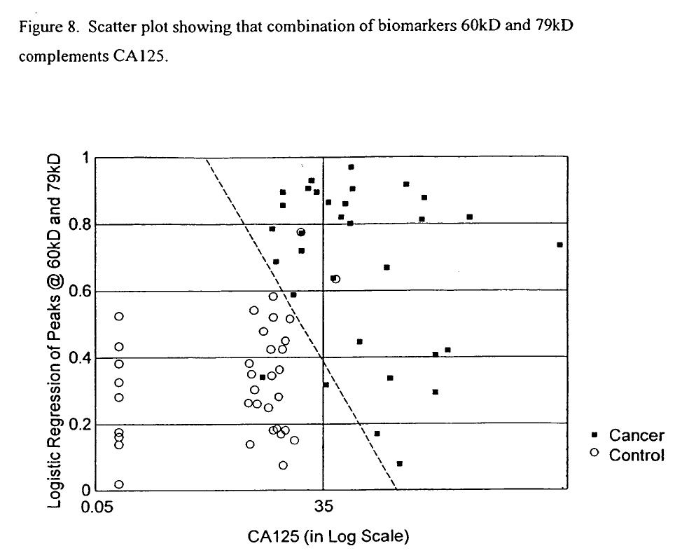

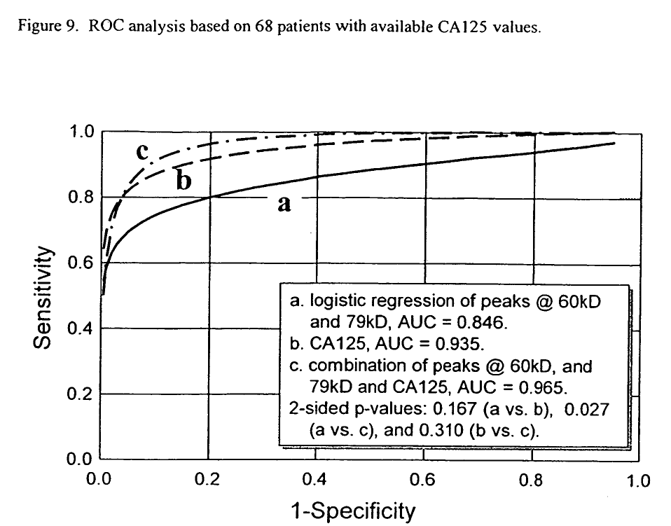

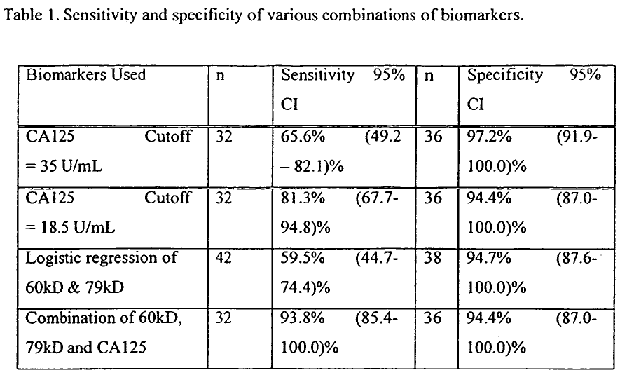

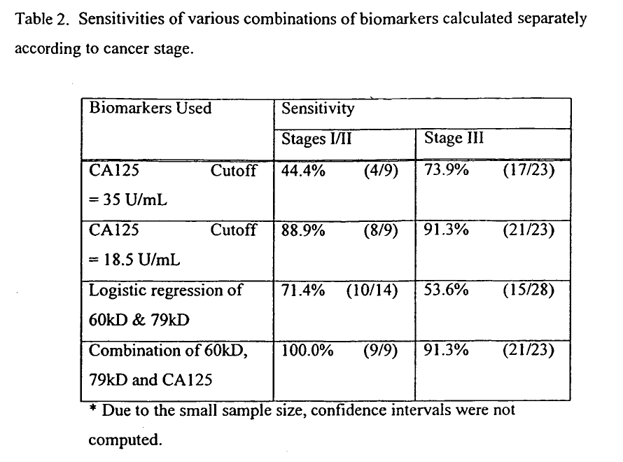

Four peaks (9.2 kD, 54 kD, 60 kD, and 79 kD) were actually used in the final statistical evaluation of diagnostic performance. They were selected for their relative high scores in UMSA analysis. The performance of individual peaks was compared to that from the logistic regression functions of all four peaks and two of the peaks (60 kD and 79 kD) using ROC analysis (FIG. 7). In the scatter plot (FIG. 8), the y-axis represents the combination of 60 kD and 79 kD through a logistic regression function. The x-axis is the CA125 value in logarithmic scale with the recommended cutoff value at 35U/mL marked as a vertical line. The dashed line shows that by combining the two biomarkers with CA125, a much improved separation between the two groups of patients can be achieved than using CA125 alone. Based on this observation, ROC analysis was performed using 68 patients with available CA125 values to compare the diagnostic performance of the combination of 60 kD and 79 kD, CA125, and the combination of all three markers (FIG. 9). The addition of the two biomarkers improves the overall performance of CA125.

Table 1 compares the estimated sensitivities and specificities of (1) CA125 alone at two different cutoff values; (2) logistic regression function of 60 kD and 79 kD, and (3) an diagnostic index which is the linear combination of (1) and (2). In the table, the first cutoff value of CA125 was the recommended value of 35U/mL. The second value at 1 8.5U/mL was selected such that CA125 achieves maximum efficiency based on ROC analysis. This resulted in a specificity of 94.4%. The remaining comparison, performed using this set specificity, indicates that the diagnostic index from the combination of the two biomarkers and CA125 improves the sensitivity from 81.3% to 93.8%. Finally, in Table 2, test sensitivities were calculated separately according to early and late disease stages. The result shows that the diagnostic index from combining the two biomarkers and CA125 retains a high level of sensitivity for the early stage cancer patients. The mean and standard deviation of the diagnostic index in the cancer group were 0.400 and 0.037, respectively, and in the non-cancer group were 0.285 and 0.620, respectively. The difference was highly significant (p<0.000001).

The invention has been described in detail with reference to particular embodiments thereof. However, it will be appreciated that those skilled in the art, upon consideration of this disclosure, may make modifications and improvements within the spirit and scope of the invention.

1-2. (canceled)

3. A method for detection and diagnosis of ovarian cancer comprising:
measuring at least one protein biomarkers in a subject sample, wherein the protein markers are selected from:

| | |
|---|---|
| Marker I: | having a molecular weight of about 8.6 kD |
| Marker II: | having a molecular weight of about 9.2 kD |
| Marker III: | having a molecular weight of about 19.8 kD |
| Marker IV: | having a molecular weight of about 39.8 kD |
| Marker V: | having a molecular weight of about 54 kD |
| Marker VI: | having a molecular weight of about 60 kD |
| Marker VII: | having a molecular weight of about 79 kD |

and; correlating the measurement of one or more protein biomarkers with a diagnosis of ovarian cancer.

4. The method of claim 3 wherein one or more protein biomarkers are used to diagnose ovarian cancer.

5. The method of claim 3 wherein a plurality of the biomarkers are detected.

6-8. (canceled)

9. The method of claim 3 wherein a single biomarker is used in combination with one or more known cancer biomarkers for diagnosing cancer.

10. The method of claim 3 wherein a plurality of the markers are used in combination with one or more known cancer markers for diagnosing cancer.

11. The method of claim 9 or 10 wherein the known cancer markers are ovarian cancer markers for diagnosing ovarian cancer.

12. The method of 11 wherein the known ovarian cancer marker is CA 125.

13-33. (canceled)

34. The method of claim 3 wherein one or more of the markers are detected using laser desorption/ionization mass spectrometry, comprising:
providing a probe adapted for use with a mass spectrometer comprising an adsorbent attached thereto, and;
contacting the subject sample with the adsorbent, and;
desorbing and ionizing the marker or markers from the probe and detecting the deionized/ionized markers with the mass spectrometer.

35-38. (canceled)

39. The method of claim 3 wherein at least one or more protein biomarkers are detected using immunoassays.

40. A process for purification of a biomarker, comprising fractioning a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers.

41-45. (canceled)

46. The process of claim 40 wherein the one or more biomarkers are selected from:

| | |
|---|---|
| Marker I: | having a molecular weight of about 8.6 kD |
| Marker II: | having a molecular weight of about 9.2 kD |
| Marker III: | having a molecular weight of about 19.8 kD |
| Marker IV: | having a molecular weight of about 39.8 kD |
| Marker V: | having a molecular weight of about 54 kD |
| Marker VI: | having a molecular weight of about 60 kD |
| Marker VII: | having a molecular weight of about 79 kD |

47. A kit for aiding the diagnosis of ovarian cancer, comprising:
an adsorbent attached to a substrate, wherein the adsorbent retains one or more biomarker selected from:

| | |
|---|---|
| Marker I: | having a molecular weight of about 8.6 kD; |
| Marker II: | having a molecular weight of about 9.2 kD; |
| Marker III: | having a molecular weight of about 19.8 kD; |
| Marker IV: | having a molecular weight of about 39.8 kD; |
| Marker V: | having a molecular weight of about 54 kD; |
| Marker VI: | having a molecular weight of about 60 kD; and |
| Marker VII: | having a molecular weight of about 79 kD, and |

48-61. (canceled)

62. The method of claim 3 wherein the stage of ovarian cancer is assessed.

63. A purified protein selected from:

| | |
|---|---|
| Marker I: | having a molecular weight of about 8.6 kD; |
| Marker II: | having a molecular weight of about 9.2 kD; |
| Marker III: | having a molecular weight of about 19.8 kD; |
| Marker IV: | having a molecular weight of about 39.8 kD; |
| Marker V: | having a molecular weight of about 54 kD; |
| Marker VI: | having a molecular weight of about 60 kD; and |
| Marker VII: | having a molecular weight of about 79 kD. |

64. (canceled)

65. A composition comprising an isolated Marker II and one more biomarkers selected from Markers I, III, IV, V, VI, and VII.

66-69. (canceled)

70. A composition comprising an isolated Marker VII and one more biomarkers selected from Markers I, II, III, IV, V, and VI.

71-80. (canceled)

81. A method for qualifying ovarian cancer status in a subject comprising:
(a) measuring at least one biomarker in a sample from the subject, wherein the biomarker is selected from the group consisting of:
transferrin or a fragment thereof; or
haptoglobulin precursor protein or a fragment thereof; and
(b) correlating the measurement with ovarian cancer status.

82. The method of claim 81 wherein a plurality of the biomarkers are measured.

83. The method of claim 81 wherein a biomarker is measured that has at least about 80 percent sequence identity to transferrin.

84. The method of claim 81 wherein a biomarker is measured that has at least about 80 percent sequence identity to haptoglobulin precursor protein or fragment thereof.
